(19) United States
(12) Patent Application Publication
(10) Pub. No.: US 2008/0306478 A1
(43) Pub. Date: Dec. 11, 2008

(54) Method and Apparatus for Electrochemically Dissolving Selected Regions of Conducting Objects in Tissue

(75) Inventors: Scott H. Bradshaw, Ottawa (CA); Lloyd A. Kennedy, Kingston (CA)

Correspondence Address: SCOTT H . BRADSHAW, 228 STONEWAY DRIVE, OTTAWA, ON, K2G 6A8, CA

(73) Assignee: Scott H. Bradshaw, Ottawa (CA)

(21) Appl. No.: 11/758,780

(22) Filed: Jun. 6, 2007

(51) Int. Cl. A61B 18/12

(52) U.S. Cl. 606/45; 607/115

(57) ABSTRACT

A method and apparatus are described for selectively removing parts of a conducting object imbedded in tissue using an electrolytic process. The method includes steps for protecting the surrounding tissue from damage during the electrolytic process. Applications for the invention include sectioning surgical and/or post-mortem tissue for gross and microscopic examination, in situations where the tissue is complicated by the presence of a metal implant.

### CROSS-REFERENCE TO RELATED APPLICATIONS

Not applicable

### STATEMENT REGARDING FEDERALLY SPONSORED RESEARCH OR DEVELOPMENT

Not applicable

### BACKGROUND OF THE INVENTION

This invention relates to the removal of conducting objects from tissue, with some identified applications being for the purpose of tissue preparation for gross and microscopic examination, including surgical resection and post-mortem analysis.

Bio-implanted metallic devices such as coronary stents and surgical staples are becoming more and more ubiquitous, however they present difficulty in surgically resected specimen analysis and post-mortem analysis. At autopsy it is frequently necessary to section through the major coronary arteries at intervals along their lengths, to allow gross inspection, and occasionally microscopic analysis as well. Because of its hardness, sectioning through a stent using conventional methods causes significant damage to and/or loss of native morphology of the underlying tissue. A review of the literature relating to this problem reveals specialized methods for making thick and thin sections through metal implants (Malik, Rippstein), suitable for gross and microscopic inspection respectively, however these methods suffer several drawbacks. They are expensive, due to the requirement for specialized cutting and/or grinding tools, and specialized acrylic for sample impregnation. They are time consuming, due to the extra processing steps involved. The resulting tissue samples also suffer several technical deficiencies, including cutting artifacts, undesirably thick microscopic sections, and a reduction in the subset of chemical and immunological stains available for tissue analysis.

Review of the literature pertaining to processing methods directed towards removal of unwanted minerals from tissue reveals that acid baths are currently well known in the industry for use in dissolving calcium from tissue to facilitate sectioning. Unfortunately extrapolation of this technique to metallic devices such as stents or staples would require either an increase of exposure time, or increase in acid strength, either of which would lead to unacceptable damage to the underlying tissue.

Review of literature pertaining to electrochemical processing in medical applications reveals that electrochemical techniques are routinely used in metal processing, and specifically in stent fabrication for the purpose of creating smooth surfaces (Callol, Andreacchi). This process has never been used in the context of removal of foreign metal from tissue, however, and no technique for protecting the underlying tissue has been put forward. Other literature refers to the use of electricity in biological environments (Thapliyal), but in this case the metal implant is simply physically removed and there is no attempt to protect the surrounding tissue or preserve the tissue morphology.

This discussion relates to metallic stents, however the invention is also applicable more generally to any situation in which foreign conducting objects can advantageously be removed from tissue. One example is the removal of surgical staples: in some situations a neoplasm boundary is close to a stapled resection margin, so that it would be advantageous to preserve the underlying tissue in the region of the staple. Again, the current state of the art provides no acceptable solution.

### BRIEF SUMMARY OF THE INVENTION

The present invention is an electrochemical method to dissolve portions of a conducting object imbedded in tissue by incorporating the conducting object into an electrolytic cell as the positive electrode. The underlying tissue is protected either by careful selection of the electrolytic solution to minimize damage, or by fixing and/or impregnating the tissue with insulating material such as formalyn or wax prior to electrolysis.

A convenient apparatus is also presented in which two bladed contacting members are used to achieve the dual function of cutting through tissue and stent coating, and closing the electrical circuit. The exposed material to be dissolved can be submerged in electrolytic solution, or alternatively the solution can be applied selectively as a droplet or continuous stream to the tissue sample in the region to be dissolved. This apparatus allows rapid processing, facilitates high precision in selecting regions of metal to be dissolved, and the compactness of the apparatus allows the possibility of viewing the process microscopically.

This invention improves upon current technology in several ways, in addition to the speed and precision already mentioned. Because the foreign material is removed from the region of tissue to be sectioned, there is no limitation in tissue thickness, no sectioning artifact, and no limit to staining techniques applicable. Another improvement is in cost: the current invention requires an inexpensive current source, and makes use of pre-processing already routinely applied to tissue preparation.

### BRIEF DESCRIPTION OF THE DRAWINGS

FIG. 1 is a perspective view of one preferred embodiment of the invention, in relation to a representative object of its action: a stent embedded in tissue.

FIG. 2 is a perspective view of one preferred embodiment of the first contacting element (2) of the invention displayed in FIG. 1.

### DETAILED DESCRIPTION OF THE INVENTION

This description refers to tissue containing a metallic stent, however the same process can be used for any conducting object imbedded in tissue.

In one preferred embodiment, the tissue is fixed in formalyn, and subsequently impregnated with wax, according to well known methods. An appropriate electrolytic solution can be prepared according to well known methods in the field of electroplating. For the purpose of the present application almost any reasonable preparation is sufficient, and an example solution can be made with concentrations of 1 mole/Litre citric acid and 1 mole/Litre sodium chloride.

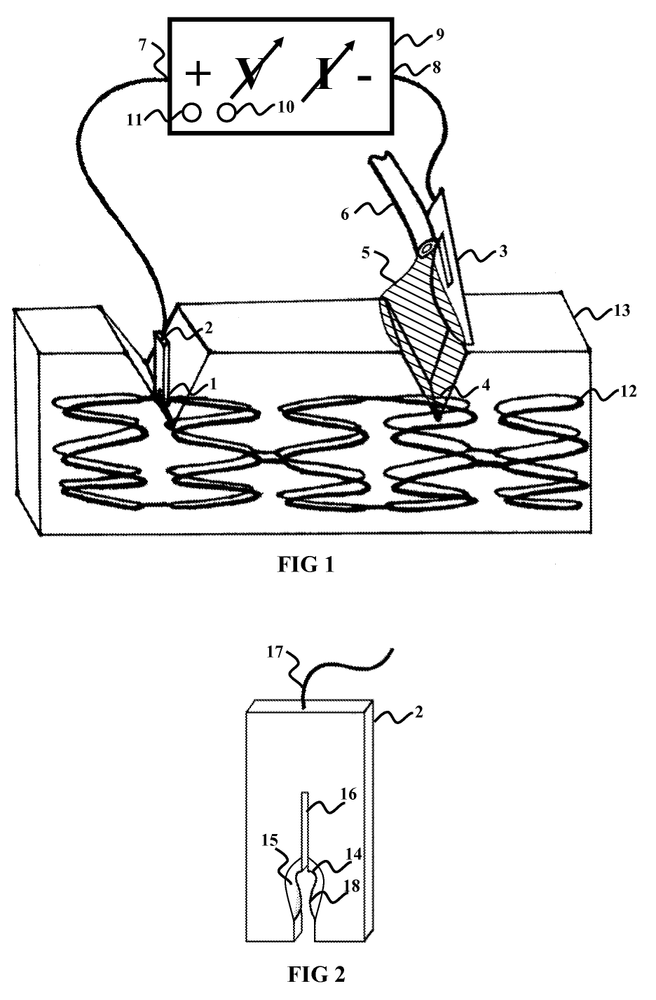

Referring to FIG. 1, the wax-impregnated tissue (13) is scored with a scalpel (3) to expose the metal stent (12), at a location (1) away from the region to be dissolved (4). A conducting spring clip (2) is attached to the exposed stent, and connected to the positive terminal (7) of a voltage source (9). The scalpel (3) is then used to score the tissue to expose the region of stent to be dissolved (4). The scalpel is then removed from contact, but held in close proximity to the exposed region. The scalpel is also attached to the negative terminal (8) of a voltage source (9). A hose (6) attached to a reservoir of electrolytic solution (not shown) provides a stream of solution (5) that connects the scalpel to the region of stent to be dissolved. The hose apparatus may be attached to the scalpel (3) for greater convenience.

FIG. 2 shows a larger view of the metal clip (2) from FIG. 1. This clip is based on a well-known method of breaking the insulation of an insulated wire, establishing electrical contact, and maintaining electrical contact with the wire, and is ubiquitous in the electronics industry as a means to crimp a connector onto a ribbon cable, for example. A bladed inner surface (18) can be used to score the stent, and a widened area (14) provides a seat for the wire to sit, and maintain electrical contact to the clip. A groove (16) allows the metal to flex, so that a wire can be scored by the higher pressure while passing by the blade (18), and flex back to maintain electrical contact when the wire is in the wider seat (14). A wire (17) is also attached to provide access to a voltage source. Additionally, the clip can be insulated, apart from the contact region (15), to protect against accidental contact with the electrolyte during the dissolving process, which would result in corrosion of the clip.

Referring back to FIG. 1, An audio (10) or visual (11) signal proportional to the amount of current draw can be incorporated, which verifies the electrical circuit has been completed at the time the stent is scored, and provides constant monitoring of the rate of reaction during the electrolysis to follow. The embodiment in FIG. 1 includes a variable voltage, which can be used to control the rate of reaction. A current limiter is also included, to limit the current flow during the periods of short circuit when the scalpel is in contact with the stent.

The scalpel can be used to repeatedly score the tissue during the electrolytic process to remove debris and renew electrical contact, and the scalpel can also be used to expose other regions of the stent. In the case illustrated in FIG. 1, a cross-section of the tissue through the stent can be obtained, provided a complete electrical path is maintained between the positive lead and the additional regions to be dissolved. If the electrical path is dissolved, a complete circuit can be re-established by re-locating the spring clip (2) to a new location along the stent.

The electrolytic reaction can be observed by eye or microscopically, and the voltage and current limits can be adjusted to increase or decrease the rate of reaction as desired. Once the tissue has been completely separated, additional corrosion time can be added to ensure that the metal dissolves sufficiently far into the wax to enable microtome sectioning of the exposed face to take place, without risk of encountering un-dissolved residual stent.

Another embodiment contacts one stent location by scratching away tissue and stent coating, and contacting with a wire twisted onto the stent, effectively replacing the function of element (2) in FIG. 1. The contact point is then insulated, with wax, varnish, or other insulating material. A score is made through the tissue and stent coating at location(s) where metal removal is desired, and the sample is then placed in an ultrasonic bath whose container is grounded. Positive voltage is applied to the stent contact wire, and the sample can be left indefinitely in the ultrasonic bath. This embodiment is intended to remove a larger volume of material, or even achieve near complete removal, depending on stent geometry.

Another embodiment provides a method to remove the metal from tissue that is not wax impregnated, for the case where rapid tissue analysis is required, or for removal of metal from living tissue. In this case the osmolality of the electrolytic solution is chosen to be isotonic to the tissue, and a mild acid (6<pH <7.4, for example), is used. In this case, a high voltage is desired, to maximize metal corrosion and therefore minimize exposure time of living tissue to acidic solution.

What is claimed is:

1. A method of partially or completely dissolving a conducting object in contact with tissue, with little or no damage to the surrounding tissue by:
applying a positive voltage to the object,
exposing the region to be dissolved to an electrolytic solution which is in turn connected to a negative potential relative to the conducting object, and
protecting the surrounding tissue from damage due to the electrolytic solution.

2. The method of claim (1) in which the tissue damage is minimized by:
adjusting the electrolytic solution pH to be close to physiologic pH; 6<pH<7.4, adjusting the electrolytic solution salinity to be isotonic to the tissue, limiting the current density of the conducting circuit so that thermal heating of the surrounding tissue results in a temperature change less than 4 degrees Celsius, and minimizing the tissue exposure time to the electrolytic solution by maximizing the current density within the limits of the prescribed pH, salinity and temperature outlined above.

3. The method of claim (1) in which the tissue damage is minimized by chemically and/or electrically insulating the tissue from the electrolytic solution prior to dissolving the conducting object.

4. The method of claim (3) in which tissue insulation is achieved by impregnating the tissue with with wax.

5. The method of claim (3) in which the electrolytic solution is acidified to a pH<3.

6. The method of claim (1) in which the tissue has been fixed with a fixative.

7. The method of claim (1) in which the electrolytic solution is agitated.

8. The method of claim (7) in which the agitation method is selected from ultrasonic, stirrer, water jet, or air jet.

9. The method of claim (1) in which the conducting object is chosen from: metallic stent, surgical staple, surgical wire, bullet or other shrapnel, prosthetic device, electrical lead, or pacemaker lead.

10. The method of claim (1) in which the exposed conducting region to be dissolved is chosen in such a way as to facilitate partial or complete sectioning of the sample, suitable for gross or microscopic examination of the underlying tissue.

11. The method of claim (1) in which the exposed conducting region is chosen so that material electrically distal to the positive contact point is dissolved initially and the dissolution continues towards the point of positive contact, so as to preserve the conducting pathway throughout the dissolution process.

12. A method of repetitively applying the method of claim (1) to a conducting object, exposing different regions to be dissolved in each successive application.

13. The method of claim (12) in which regions electrically distal to the point of positive contact are exposed initially, and subsequent exposures are electrically more proximal to the point of positive contact, so as to preserve the conducting pathway throughout the dissolution process.

14. The method of claim (12) in which the positive voltage is re-applied to a different part of the imbedded conducting object between exposures.

15. The method of claim (1) in which an electrically conducting bladed member is used to score and maintain contact with the conducting object, and subsequently used to apply a voltage.

16. The method of claim (1) in which the region of the conducting object to be dissolved is exposed by scoring the selected region of the object with a knife blade.

17. The method of claim (16) in which the part of the conducting object to be dissolved is repeatedly scored to remove debris and expose new conducting area during the dissolution process.

18. The method of claim (1) in which the region of the conducting object to be dissolved is exposed to electrolytic solution by immersing the region in solution.

19. The method of claim (1) in which the region of the conducting object to be dissolved is exposed to electrolytic solution by applying a drop of electrolytic solution or a stream of electrolytic solution to the area, and touching the negative lead to the droplet or stream.

20. The method of claim (19) in which the area to be dissolved is visualized microscopically while the dissolution takes place.

21. An apparatus for realizing one embodiment of the method in (1) comprising:
a hose attached to a reservoir of electrolytic solution which delivers a continuous stream of solution to the region of the conducting object to be removed, which serves the triple purpose of: i) completing the electrical circuit necessary for the electrolytic cell, ii) replenishing the electrolytic solution, and iii) agitating the reaction site to facilitate removal of debris,
a first sharpened member, attached to the positive lead of a voltage source, which serves the dual purpose of: i) cutting into the tissue preparation and protective coating (if present) of the conducting object, and ii) establishing electrical contact to the conducting object, and
a second sharpened member, attached to the negative lead of a voltage source, which serves the triple function of i) cutting into the tissue preparation and protective coating (if present) of the conducting object, ii) establishing electrical contact to the electrolytic solution overlying the conducting object, and iii) periodically removing debris from the exposed conducting object.

22. The apparatus of claim (21) in which the first sharpened member is insulated apart from the contact point to the conducting object.

23. The apparatus of claim (21) in which the hose and continuous stream are replaced with a dropper, which is used to discontinuously refresh the electrolytic solution.

24. The apparatus of claim (21) in which the first sharpened member is a flexible metallic gripper, with sharpened internal edges for scoring and maintaining contact with the conducting object.

25. The apparatus of claim (21) in which the voltage source is adjustable, and current limited with the current limit being adjustable as well.

26. The apparatus of claim (21) in which an audio or visual cue is elicited when a current threshold is exceeded, signaling that the electrical circuit has been closed, with the cue intensity or volume being fixed, or proportional to the magnitude of current draw, and the cue threshold and intensity ramp both being adjustable.
